(19) United States
(12) Patent Application Publication
(10) Pub. No.: US 2020/0315580 A1
(43) Pub. Date: Oct. 8, 2020

(54) SYSTEM AND METHOD OF ALIGNING A CENTER OF A BLOOD VESSEL DURING ULTRASOUND IMAGING AND PROCEDURE GUIDANCE

(71) Applicant: Melvyn Lewis Harris, Folsom, CA (US)

(72) Inventor: Melvyn Lewis Harris, Folsom, CA (US)

(21) Appl. No.: 16/841,179

(22) Filed: Apr. 6, 2020

Related U.S. Application Data
(60) Provisional application No. 62/829,703, filed on Apr. 5, 2019.

(51) Int. Cl. A61B 8/00; A61B 8/08

(52) U.S. Cl. CPC A61B 8/463; A61B 8/0891; A61B 8/4254; A61B 8/5223; A61B 8/4444; A61B 8/469

(57) ABSTRACT

A system and method for determining a center of a blood vessel. An ultrasonic probe includes a midline marker. A computing system includes a processor, a memory, a user interface, and an imaging screen. The processor displays imagery from the ultrasonic probe on the imaging screen, receives a selection of a desired blood vessel displayed on the imaging screen via the user interface, determines a center of the desired blood vessel, and produces an alert when the midline marker of the ultrasonic probe is disposed over the center of the desired blood vessel.

### CROSS-REFERENCE TO RELATED APPLICATION

This application claims the benefit of priority of U.S. provisional application No. 62/829,703, filed Apr. 5, 2019, the contents of which are herein incorporated by reference.

### BACKGROUND OF THE INVENTION

The present invention relates to finding a center of a blood vessel and, more particularly, to method of determining an optimal alignment of an ultrasound probe with a center of desired blood vessel.

Healthcare providers use ultrasound imaging to obtain blood vessel access, with a needle or catheter when encountering the “difficult to access patient.” These are patients whose blood vessels are not either seen with the unaided eye or are palpable. This procedure requires very precise alignment of the middle of the ultrasound probe with the center of the desired blood vessel. The problem is that the healthcare worker presently only uses their individual eye hand coordination to align the middle of the probe with the center of the blood vessel. This method of alignment is problematic, differs from one individual to another, and can lead to failure including multiple failed attempts and possible damage to the blood vessel.

As can be seen, there is a need for a method of determining an optimal alignment of an ultrasound probe with a center of a desired blood vessel.

### SUMMARY OF THE INVENTION

In one aspect of the present invention, a method of determining a center of a blood vessel comprises steps of: pressing an ultrasonic probe against a skin of a patient, wherein the ultrasonic probe comprises a midline marker; selecting a desired blood vessel on an imaging screen via a user interface of a computing system, the imaging screen displaying video imagery from the ultrasonic probe, the computing system comprising a processor and a memory, wherein the processor determines a center of the desired blood vessel; and produces an alert when the midline marker of the ultrasonic probe is disposed over the center of the desired blood vessel.

In another aspect of the present invention, a system for determining a center of a blood vessel comprises: an ultrasonic probe comprising a midline marker; and a computing system comprising a processor, a memory, a user interface, and an imaging screen, wherein the processor displays imagery from the ultrasonic probe on the imaging screen, receives a selection of a desired blood vessel displayed on the imaging screen via the user interface, determines a center of the desired blood vessel; and produces an alert when the midline marker of the ultrasonic probe is disposed over the center of the desired blood vessel.

These and other features, aspects and advantages of the present invention will become better understood with reference to the following drawings, description and claims.

### BRIEF DESCRIPTION OF THE DRAWINGS

FIG. 1 is an illustration of an ultrasound computer imaging screen with a vertical midline marker A-B;

FIG. 2 is an illustration of an ultrasound probe with a midline marker C-D;

FIG. 3 is an illustration of a selection of a blood vessel and determination of its diameter E-F;

FIG. 4 is an illustration of a calculation of the blood vessel center (X) by determining a radius (R);

FIG. 5 is an illustration of a calculation of a blood vessel center (X) to vertical midline market E′; and

FIG. 6 is an illustration of an alignment with a blood vessel.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes a system and method for aligning the center of a blood vessel during ultrasound imaging and procedure guidance. Presently all ultrasound guided blood vessel access is achieved by simple human eye hand coordination between the image as seen on the imaging screen, the probe, and desired blood vessel. The present invention precisely aligns the middle of the probe with the center of the desired blood vessel there-by eliminating individual differences in eye hand coordination. By precisely aligning the center of desired blood vessel to the exact middle of the probe the present invention ensures that optimal alignment is achieved with respect to inserting a needle or catheter into the desired blood vessel.

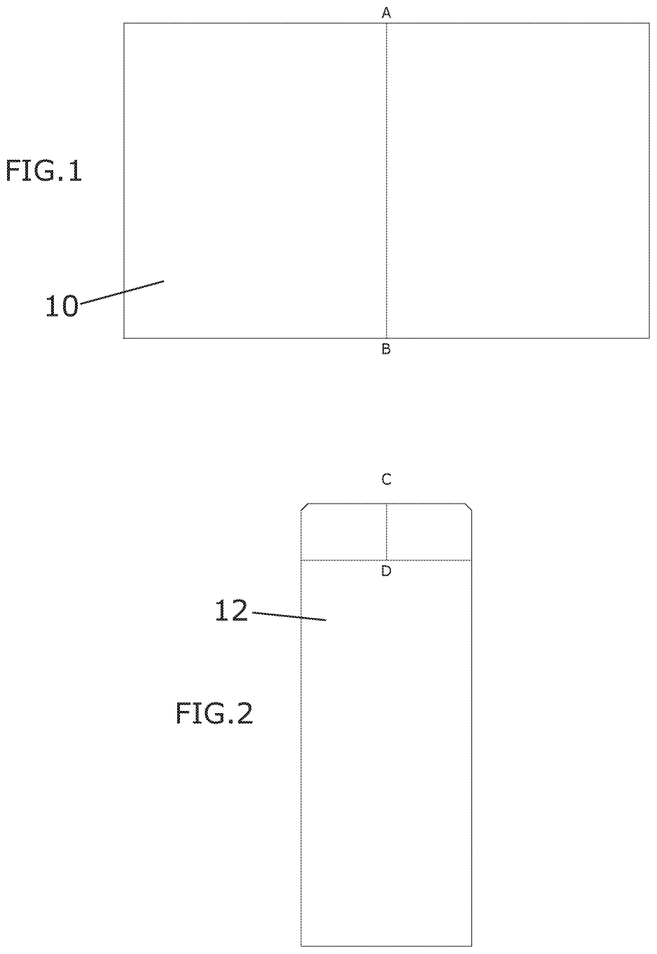

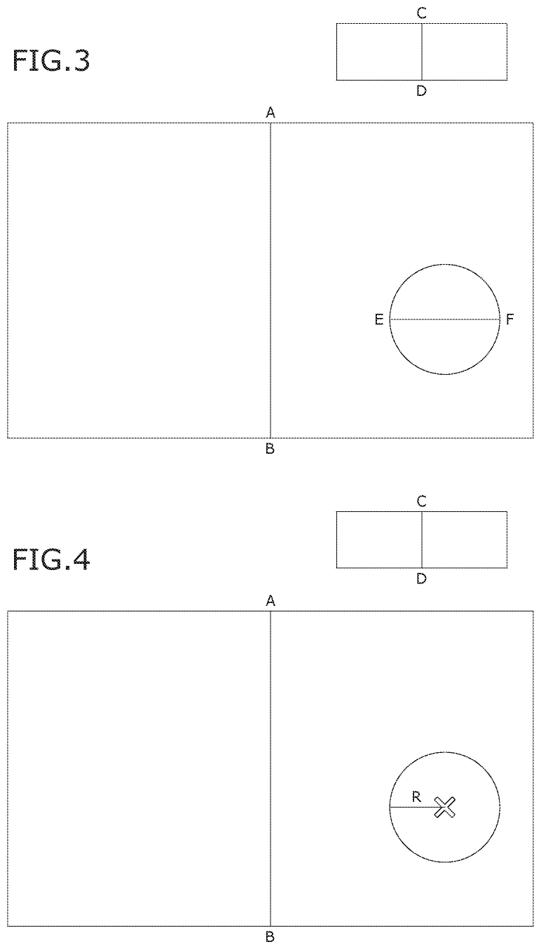

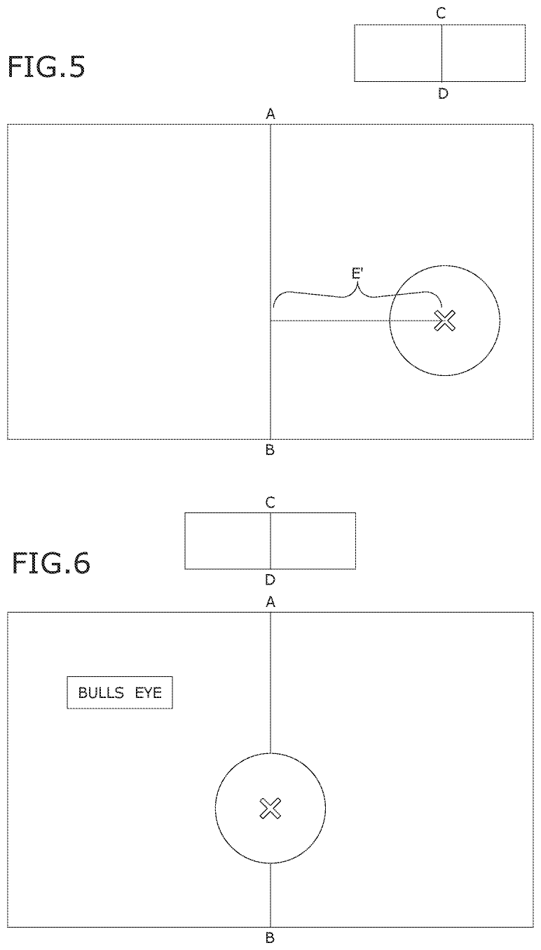

Referring to FIGS. 1 through 6, the present invention includes a computing system, an ultrasonic probe 10, a user interface, and an imaging screen 12. The ultrasonic probe 10 is a transducer that produces sound waves that bounce off body tissues and make echoes. The transducer also receives the echoes and sends them to the computing system that uses them to create an image on the imaging screen 12 called a sonogram. The ultrasonic probe 10 may include a midline marker C-D that indicates a center location of the probe tip. The imaging screen 12 may also include a vertical midline A-B that indicates a center location of the graphic user interface and corresponds with the midline marker C-D of the ultrasonic probe 10.

The computing system processes, renders, and displays image data captured by the ultrasonic probe 10. The computing system is at least a processor and the memory. The computing system may execute on any suitable operating system such as IBM's zSeries/Operating System (z/OS), MS-DOS, PC-DOS, MAC-iOS, WINDOWS, UNIX, OpenVMS, ANDROID, an operating system based on LINUX, or any other appropriate operating system, including future operating systems.

In particular embodiments, the computing system includes a processor, memory, a user interface, and a communication interface. In particular embodiments, the processor includes hardware for executing instructions, such as those making up a computer program. The memory includes main memory for storing instructions such as computer program(s) for the processor to execute, or data for processor to operate on. The memory may include an HDD, a floppy disk drive, flash memory, an optical disc, a magneto-optical disc, magnetic tape, a Universal Serial Bus (USB) drive, a solid-state drive (SSD), or a combination of two or more of these. The memory may include removable or non-removable (or fixed) media, where appropriate. The memory may be internal or external to the computing system, where appropriate. In particular embodiments, the memory is non-volatile, solid-state memory.

The user interface includes hardware, software, or both providing one or more interfaces for user communication with the computing system. As an example and not by way of limitation, the user interface may include a keyboard, keypad, microphone, monitor, mouse, printer, scanner, speaker, still camera, stylus, tablet, touchscreen, trackball, video camera, another user interface or a combination of two or more of these.

The communication interface includes hardware, software, or both providing one or more interfaces for communication (e.g., packet-based communication) between the computing system, the ultrasonic probe 10, and other computing systems or one or more networks. The ultrasonic probe 10 may be directly hard wired to the computing system 100, such as through a USB port or other cable connection interface and may transfer image data through the cable connection. Alternatively, the ultrasonic probe 10 may transfer image data using wireless communication. As an example, and not by way of limitation, the computing system and the ultrasonic probe 10 may include a communication interface including a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface. As an example and not by way of limitation, the ultrasonic probe 10 and the computing system may communicate via an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, the ultrasonic probe 10 and the computing system 100 may communicate via a wireless PAN (WPAN) (e.g., a BLUETOOTH WPAN), a WI-FI network, a WI-MAX network, a cellular telephone network (e.g., a Global System for Mobile Communications (GSM) network), or other suitable wireless network or a combination of two or more of these. The ultrasonic probe 10 and the computing system may include any suitable communication interface for any of these networks, where appropriate.

The ultrasonic probe 10 provides video imagery to the computing system and an algorithm which processes the video to identify where the approximate vessel center is located. To determine the center of the blood vessel, the ultrasonic probe 10 is pressed against a skin of a patient. Blood vessels may appear on the imaging screen 12. A desired blood vessel is then selected. The desired blood vessel may be selected by a point and click action by a mouse, by tapping the imaging screen 12 using a touch screen, or by other actions facilitated by the user interface. The computing system determines the diameter of the desired blood vessel by using the computer calipers. This measurement is divided in half by the computing system. This resulting calculation represents the radius of the blood vessel which is a line drawn from the center of the blood vessel to its periphery. The present invention marks the center of the blood vessel as indicated in FIG. 4. The computing system next calculates the distance from the imaging screens vertical midline marker A-B to the calculated center of the blood vessel.

The user may then move the ultrasonic probe 10 so that the desired blood vessel moves towards the vertical midline marker A-B of the imaging screen 12. When the center of the desired blood vessel is brought toward the imaging screen's vertical midline marker A-B, the computing system prompts an alert to the health care practitioner when the imaging screens vertical midline marker A-B is passing directly though the center of the desired blood vessel. When this alignment is achieved, the middle of the probe, the probe's midline marker C-D is directly over the center of the blood vessel. Upon achieving this alignment, the computing system notifies the medical provider by the alert, which may include a visual alert on the imaging screen 12, an audio alert, a vibration alert, or combination thereof.

For the purpose of this invention it is understood that when referring to a blood vessel one is referring to a circular structure. The present invention uses the ultrasound probe 10 with a midline marker and a computing system and imaging screen. The computing system calculates assorted geometrical measurements such as lengths of lines, diameter of a circle, radius of a circle and to make further calculations from these measurements using the geometrical relationships between diameter (D), radius (R), circumference (C), and the constant Pi (3.14) as embodied in the formula C=D multiplied by 3.14 and R=D/2. Additional calculations to determine the diameter of the blood vessel, then its radius, and thereby its center can be obtained by measuring the circumference of the blood vessel and then calculating the diameter by dividing the circumference by Pi (3.14)

By allowing the computer operating system to make several simple geometrical calculations and coordinating these calculations with the vertical midline of the imaging screen and the middle of the ultrasound probe the computer is able to standardize displaying the center of the blood vessel in alignment with the middle of the ultrasound probe for all medical providers regardless of their own eye hand coordination capabilities.

It should be understood, of course, that the foregoing relates to exemplary embodiments of the invention and that modifications may be made without departing from the spirit and scope of the invention as set forth in the following claims.

What is claimed is:

1. A method of determining a center of a blood vessel comprising steps of:
pressing an ultrasonic probe against a skin of a patient, wherein the ultrasonic probe comprises a midline marker;
selecting a desired blood vessel on an imaging screen via a user interface of a computing system, the imaging screen displaying video imagery from the ultrasonic probe, the computing system comprising a processor and a memory, wherein the processor
determines a center of the desired blood vessel, and
produces an alert when the midline marker of the ultrasonic probe is disposed over the center of the desired blood vessel.

2. The method of claim 1, wherein the imaging screen comprises a vertical midline marker that corresponds with the midline marker of the ultrasonic probe.

3. The method of claim 2, wherein the processor further
determines a distance from the center of the desired blood vessel and the vertical midline marker of the imaging screen to produce the alert when the midline marker of the ultrasonic probe is moved over the center of the desired blood vessel.

4. The method of claim 3, wherein the processor determines a center of the desired blood vessel by
determining a diameter of the desired blood vessel using computer calipers,
determining a radius of the desired blood vessel by dividing the diameter in half, and
marking the center of the desired blood vessel, wherein a marking of the center of the desired blood vessel is produced on the imaging screen.

5. The method of claim 1, wherein the alert is at least one of a visual alert on the imaging screen, an audio alert and a vibration alert.

6. A system for determining a center of a blood vessel comprising:
an ultrasonic probe comprising a midline marker; and
a computing system comprising a processor, a memory, a user interface, and an imaging screen, wherein the processor
displays imagery from the ultrasonic probe on the imaging screen,
receives a selection of a desired blood vessel displayed on the imaging screen via the user interface,
determines a center of the desired blood vessel, and
produces an alert when the midline marker of the ultrasonic probe is disposed over the center of the desired blood vessel.

7. The system of claim 6, wherein the imaging screen comprises a vertical midline marker that corresponds with the midline marker of the ultrasonic probe.

8. The system of claim 7, wherein the processor further
determines a distance from the center of the desired blood vessel and the vertical midline marker of the imaging screen to produce the alert when the midline marker of the ultrasonic probe is moved over the center of the desired blood vessel.

9. The system of claim 8, wherein the processor determines a center of the desired blood vessel by
determining a diameter of the desired blood vessel using computer calipers,
determining a radius of the desired blood vessel by dividing the diameter in half, and
marking the center of the desired blood vessel, wherein a marking of the center of the desired blood vessel is produced on the imaging screen.

10. The system of claim 6, wherein the alert is at least one of a visual alert on the imaging screen, an audio alert and a vibration alert.
